(19) United States
(12) Patent Application Publication
(10) Pub. No.: US 2025/0282715 A1
(43) Pub. Date: Sep. 11, 2025

(54) SOLID STATE FORMS OF MIRDAMETINIB AND PROCESS FOR PREPARATION THEREOF

(71) Applicant: Assia Chemical Industries Ltd., Tel Aviv (IL)

(72) Inventors: Nikolina Janton, Jakovlje (HR); Zvonimir Perkovic, Zagreb (HR); Helena Ceric, Zagreb (HR)

(21) Appl. No.: 18/858,794

(22) PCT Filed: May 16, 2023
(86) PCT No.: PCT/IB2023/055031

§ 371 Date: Oct. 22, 2024

Related U.S. Application Data
(60) Provisional application No. 63/342,658, filed on May 17, 2022.

(51) Int. Cl. C07C 259/10; A61K 31/21; C07C 55/07

(52) U.S. Cl. CPC C07C 259/10; A61K 31/21; C07C 55/07

(57) ABSTRACT

The present disclosure encompasses solid state forms of Mirdametinib, particularly to Mirdametinib complexes. In embodiments the present disclosure encompasses processes for preparation thereof, and pharmaceutical compositions thereof.

### FIELD OF THE DISCLOSURE

The present disclosure encompasses solid state forms of Mirdametinib, particularly to Mirdametinib complexes. In embodiments the present disclosure encompasses processes for preparation thereof, and pharmaceutical compositions thereof.

### BACKGROUND OF THE DISCLOSURE

Mirdametinib has the chemical name N-[(2R)-2,3-dihydroxypropoxyl]-3,4-difluoro-2-[(2-fluoro-4-iodophenyl)amino]benzamide and the following chemical structure:

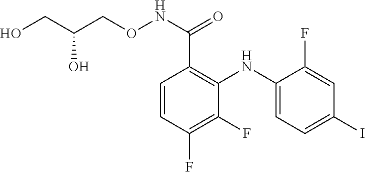 [SMILES: O=C(NOC[C@H](O)CO)c1ccc(F)c(F)c1Nc1ccc(I)cc1F]

Mirdametinib is a MEK inhibitor which is investigated and developed for the treatment of patients with Neurofibromatosis Type 1 Associated Plexiform Neurofibromas and for the treatment of patients with low-grade gliomas. Mirdametinib is also under investigation for the treatment of metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma.

Mirdametinib, as well as its crystalline forms I and II, are described in International Publication No. WO 2002/006213.

International Publication No. WO 2005/040098, and U.S. Pat. Nos. 11,066,358 and 11,453,641 describe crystalline form IV of Mirdametinib. International Publication No. WO 2022/177556 describes crystalline form IV of Mirdametinib which is essentially pure.

International Publication No. WO 2007/042885 describes crystalline forms I, II and IV of Mirdametinib.

International Publication No. WO 2022/177557 and U.S. Pat. No. 11,084,780 describe additional polymorphs of Mirdametinib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Mirdametinib, as well as complexes and salts of Mirdametinib and their solid state forms.

### SUMMARY OF THE DISCLOSURE

The present disclosure encompasses solid state form of Mirdametinib, particularly to Mirdametinib complexes. In embodiments the present disclosure encompasses processes for preparation thereof, and pharmaceutical compositions thereof.

These solid state forms of the present disclosure, particularly crystalline complexes of Mirdametinib can be used to prepare other solid state forms of Mirdametinib, Mirdametinib salts or co-crystals and their solid state forms.

The present disclosure also provides uses of the said solid state form of Mirdametinib complexes in the preparation of other solid state forms of Mirdametinib, or Mirdametinib salts or co-crystals salts thereof.

The present disclosure provides solid state forms, particularly crystalline complexes of Mirdametinib, for use in medicine, including for the treatment of: patients with Neurofibromatosis Type 1 Associated Plexiform Neurofibromas, low-grade gliomas, metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably Neurofibromatosis Type 1 Associated Plexiform Neurofibromas or low-grade gliomas.

The present disclosure also encompasses the use of the solid state forms, particularly crystalline complexes of Mirdametinib of the present disclosure for the preparation of pharmaceutical compositions and/or formulations. In embodiments, solid state forms, particularly crystalline complexes of Mirdametinib of the present disclosure are used to prepare oral dosage forms of Mirdametinib and of Mirdametinib salts or co-crystals.

In another aspect, the present disclosure provides pharmaceutical compositions, such as oral dosage forms of crystalline complexes of Mirdametinib, comprising the solid state forms, particularly crystalline complexes of Mirdametinib according to the present disclosure.

The present disclosure includes processes for preparing the above mentioned pharmaceutical compositions. The processes include combining any one or a combination of the solid state forms, particularly crystalline complexes of Mirdametinib with at least one pharmaceutically acceptable excipient.

The solid state forms, particularly crystalline complexes of Mirdametinib as defined herein and the pharmaceutical compositions or formulations of solid state forms, particularly crystalline complexes of Mirdametinib may be used as medicaments, such as for the treatment of patients with Neurofibromatosis Type 1 Associated Plexiform Neurofibromas and for the treatment of patients with low-grade gliomas, or for the treatment of: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably Neurofibromatosis Type 1 Associated Plexiform Neurofibromas or low-grade gliomas.

The present disclosure also provides methods of treating patients with Neurofibromatosis Type 1 Associated Plexiform Neurofibromas, and for the treatment of patients with low-grade gliomas, or for the treatment of: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably Neurofibromatosis Type 1 Associated Plexiform Neurofibromas or low-grade gliomas, by administering a therapeutically effective amount of any one or a combination of the solid state forms, particularly crystalline complexes of Mirdametinib of the present disclosure, or at least one of the above pharmaceutical compositions, to a subject suffering from Neurofibromatosis Type 1 Associated Plexiform Neurofibromas, or low-grade gliomas, or metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably to a subject suffering from Neurofibromatosis Type 1 Associated Plexiform Neurofibromas, or low-grade gliomas.

The present disclosure also provides uses of solid state forms, particularly crystalline complexes of Mirdametinib of the present disclosure, or at least one of the above pharmaceutical compositions, for the manufacture of medicaments for treating patients with Neurofibromatosis Type 1 Associated Plexiform Neurofibromas or for the treatment of patients with low-grade gliomas, or for the treatment of patients with: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably patients with Neurofibromatosis Type 1 Associated Plexiform Neurofibromas or low-grade gliomas.

### BRIEF DESCRIPTION OF THE DRAWINGS

FIG. 1 shows a characteristic X-ray powder diffraction pattern (XRPD) of Mirdametinib oxalic acid co-crystal form A.

FIG. 2 shows a characteristic FTIR spectrum of Crystalline Mirdametinib oxalic acid co-crystal form A.

FIG. 3a shows a characteristic solid-state ¹³C NMR spectrum of Crystalline Mirdametinib oxalic acid co-crystal form A (at the range from 200-0 ppm).

FIG. 3b shows a characteristic solid-state ¹³C NMR spectrum of Crystalline Mirdametinib oxalic acid co-crystal form A (at the range from 200-100 ppm).

FIG. 3c shows a characteristic solid-state ¹³C NMR spectrum of Crystalline Mirdametinib oxalic acid co-crystal form A (at the range from 100-0 ppm).

### DETAILED DESCRIPTION OF THE DISCLOSURE

The present disclosure encompasses solid state form of Mirdametinib, particularly to Mirdametinib complexes. In embodiments the present disclosure encompasses processes for preparation thereof, and pharmaceutical compositions thereof.

Solid state properties of Mirdametinib and Mirdametinib complexes and crystalline polymorphs thereof can be influenced by controlling the conditions under which Mirdametinib and crystalline polymorphs thereof are obtained in solid form.

The solid state form may be referred to herein as “Mirdametinib Form name” or “Crystalline Form name of Mirdametinib” or “Crystalline Mirdametinib Form name” or “Crystalline polymorph name of Mirdametinib” or “Crystalline Mirdametinib polymorph name” or “Mirdametinib polymorph name”. For example, crystalline Form I of Mirdametinib may be interchangeably referred to herein as Mirdametinib Form I or as Crystalline Mirdametinib Form I or as Crystalline polymorph I of Mirdametinib or as Crystalline Mirdametinib polymorph I or Mirdametinib polymorph I.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Mirdametinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Mirdametinib. In some embodiments of the disclosure, the described crystalline polymorph of Mirdametinib may contain from about 1% to about 20% (w/w), from about 5% to about 20% (w/w), or from about 5% to about 10% (w/w) of one or more other solid state forms of Mirdametinib.

Depending on which other crystalline polymorphs a comparison is made, the crystalline polymorphs of Mirdametinib of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Mirdametinib referred to herein as being characterized by graphical data “as depicted in” or “as substantially depicted in” a Figure will thus be understood to include any crystal forms of Mirdametinib characterized with the graphical data having such small variations, as are well known to the skilled person, in comparison with the Figure.

As used herein, and unless stated otherwise, the term “anhydrous” in relation to co-crystals and crystalline forms of Mirdametinib, relates to a crystalline form of Mirdametinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Mirdametinib or Mirdametinib HCl of the present disclosure corresponds to a crystalline polymorph of Mirdametinib or Mirdametinib HCl that is physically separated from the reaction mixture in which it is formed.

As used herein, unless stated otherwise, the XRPD measurements are taken using copper Kα1 radiation wavelength 1.5418 Å. XRPD peaks reported herein are measured using Cuk α1 radiation, λ=1.5418 Å, typically at a temperature of 25±3° C.

As used herein, unless stated otherwise, solid state ¹³C NMR data is obtained using ¹³C CP/MAS NMR method. Particularly, as used herein, unless stated otherwise, the ¹³C CP/MAS NMR reported herein are measured at 700 MHz, preferably at a temperature of at 293 K±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The present disclosure encompasses Mirdametinib: oxalic acid complex, preferably Mirdametinib: oxalic acid co-crystal.

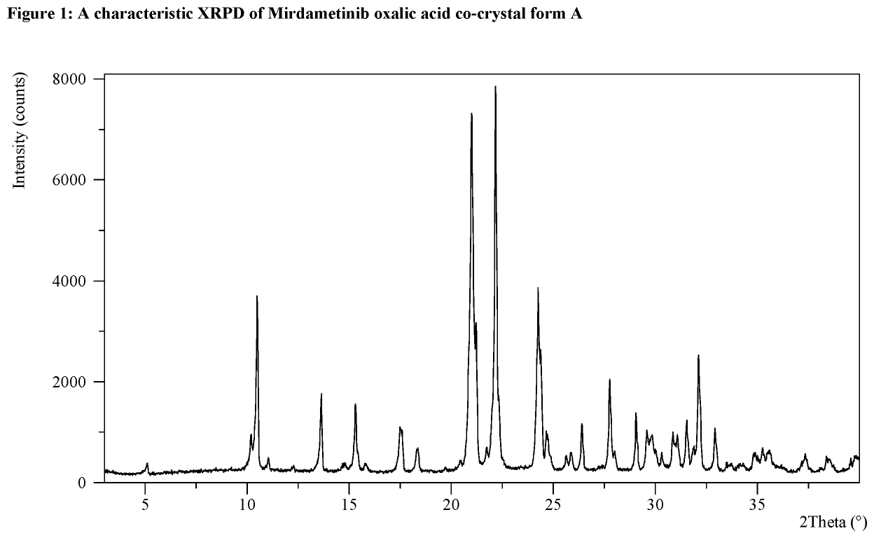

The disclosure further encompasses a crystalline form of Mirdametinib oxalic acid co-crystal, designated form A. Crystalline Form A of Mirdametinib oxalic acid co-crystal may be characterized by data selected from one or more of the following: an X-ray powder diffraction pattern substantially as depicted in FIG. 1; an X-ray powder diffraction pattern having peaks at 10.5, 13.6, 15.3, 21.2 and 22.2 degrees 2-theta±0.2 degrees 2-theta; and combinations of these data.

Crystalline Form A of Mirdametinib oxalic acid co-crystal according to any aspect or embodiment of the present disclosure may be further characterized by an X-ray powder diffraction pattern having peaks at 10.5, 13.6, 15.3, 21.2 and 22.2 degrees 2-theta±0.2 degrees 2-theta, and also having any one, two, three, four or five additional peaks selected from 5.1, 11.1, 24.3, 26.4 and 31.5 degrees 2-theta±0.2 degrees 2-theta.

Optionally, crystalline Form A of Mirdametinib oxalic acid co-crystal according to any aspect or embodiment of the present disclosure may be characterized by an X-ray powder diffraction pattern having peaks at 10.5, 13.6, 15.3, 21.2 and 22.2 degrees 2-theta±0.2 degrees 2-theta, and also having any one additional peak selected from: 5.1, 11.1, 24.3, 26.4 and 31.5 degrees 2-theta±0.2 degrees 2-theta.

Alternatively, crystalline Form A of Mirdametinib oxalic acid co-crystal according to any aspect or embodiment of the present disclosure may be characterized by an X-ray powder diffraction pattern having peaks at 5.1, 10.5, 11.1, 13.6, 15.3, 21.2, 22.2, 24.3, 26.4, and 31.5 degrees 2-theta±0.2 degrees 2-theta.

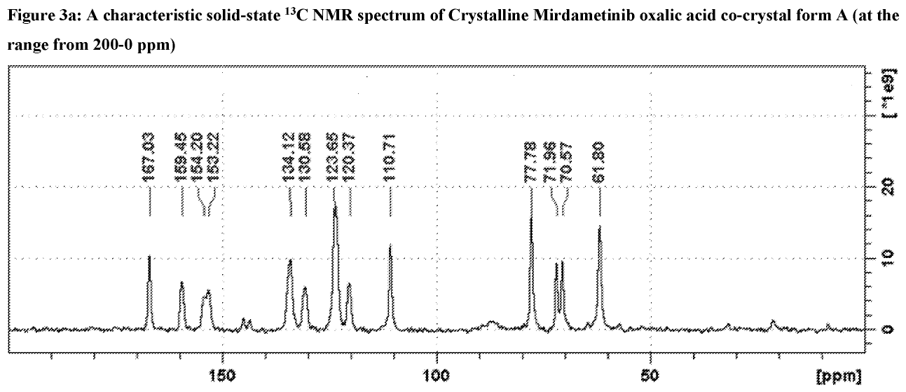

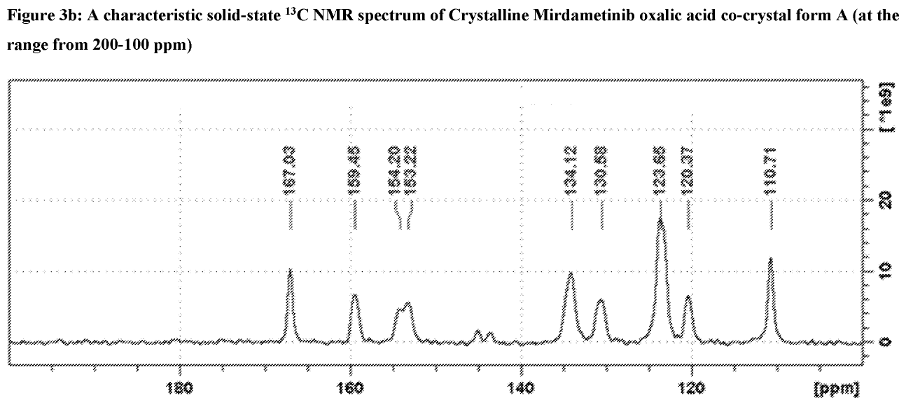

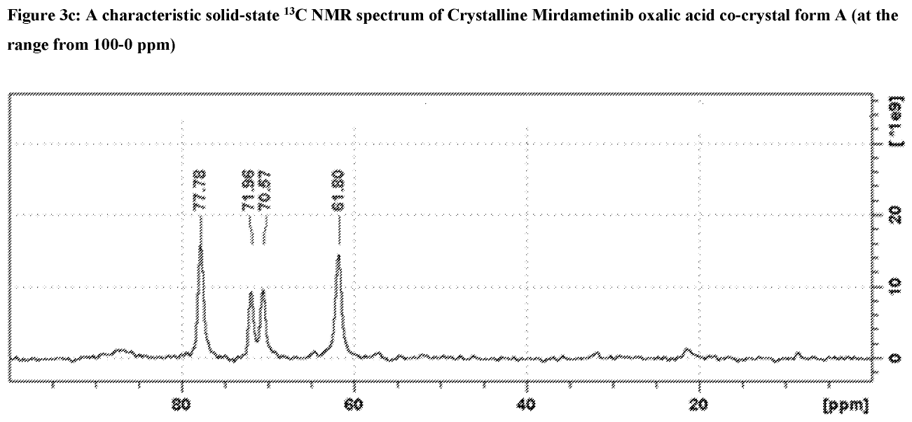

Alternatively, crystalline Form A of Mirdametinib oxalic acid co-crystal according to any aspect or embodiment of the present disclosure may be characterized by data selected from one or more of the following: a solid state ¹³C NMR spectrum with characteristic peaks at: 159.5, 134.1, 123.7 and 77.8 ppm±0.2 ppm; a solid state ¹³C NMR spectrum as depicted in any of FIG. 3a, 3b or 3c; and combinations thereof. Crystalline Form A of Mirdametinib oxalic acid co-crystal may be further characterized by a solid state ¹³C NMR spectrum having the following chemical shift absolute differences from reference peak at 61.8 ppm±1 ppm: 97.7, 72.3, 61.9 and 16.0 ppm±0.1 ppm; a solid state ¹³C NMR having the following peak list: 159.5, 134.1, 123.7 and 77.8 ppm±0.2 ppm; and combinations thereof.

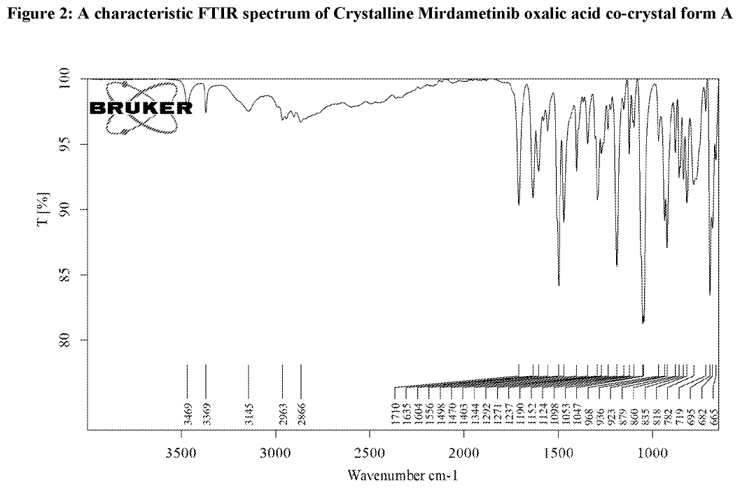

Crystalline Form A of Mirdametinib oxalic acid co-crystal according to any aspect or embodiment of the present disclosure may be further characterized by FTIR spectrum having peaks at 1710, 1498, 1190 and 1292±4 cm⁻¹; or by a FTIR spectrum substantially as depicted in FIG. 2; and combinations of these data.

In any aspect or embodiment of the present disclosure, Crystalline Form A of Mirdametinib oxalic acid co-crystal is isolated.

Crystalline Mirdametinib oxalic acid co-crystal according to any aspect or embodiment of the disclosure may be polymorphically pure or substantially free of any other forms. Preferably crystalline Mirdametinib oxalic acid co-crystal as described in any aspect or embodiment of the disclosure contains: no more than about 20%, no more than about 10%, no more than about 5%, no more than about 2%, no more than about 1% or about 0% of any other crystalline forms of Mirdametinib: oxalic acid cocrystal or Mirdametinib oxalate, more preferably wherein the crystalline Mirdametinib oxalic acid co-crystal contains: no more than about 20%, no more than about 10%, no more than about 5%, no more than about 2%, no more than about 1% or about 0% of any other solid state forms of Mirdametinib: oxalic acid cocrystal.

Crystalline Form A of Mirdametinib oxalic acid co-crystal according to any aspect or embodiment of the present disclosure may be an anhydrous form. The solvent (for example, ester solvents, such as C₄ to C₆ esters, particularly ethyl acetate, and isopropyl acetate) content in crystalline form A of Mirdametinib oxalic acid co-crystal may be: about 0.5% (w/w) or less, about 0.4% (w/w) or less, or about 0.3% (w/w), as determined by GC analysis.

Crystalline Form A of Mirdametinib oxalic acid co-crystal according to any aspect or embodiment of the present disclosure is non-hygroscopic form, showing little or no water absorption at up to 90% RH (at a temperature of about 25° C.).

Crystalline Form A of Mirdametinib oxalic acid co-crystal may be characterized by each of the above characteristics alone or by all possible combinations, for example., an XRPD pattern having peaks at 10.5, 13.6, 15.3, 21.2 and 22.2 degrees 2-theta±0.2 degrees 2-theta; an XRPD pattern as depicted in FIG. 1; or a solid state a solid state ¹³C NMR spectrum as depicted in any of FIG. 3a, 3b or 3c; and combinations thereof.

The above solid state forms can be used to prepare other crystalline polymorphs of Mirdametinib, Mirdametinib salts or co-crystals and their solid state forms.

The present disclosure encompasses a process for preparing other solid state forms of Mirdametinib, Mirdametinib salts or co-crystals and their solid state forms thereof. The process includes preparing any one of the solid state forms of Mirdametinib or Mirdametinib complexes by the processes of the present disclosure, and converting that form to a different form of Mirdametinib, Mirdametinib salt or co-crystal and solid state forms thereof.

The present disclosure provides the above described solid state forms, particularly crystalline complexes of Mirdametinib, for use in the preparation of pharmaceutical compositions comprising Mirdametinib and/or crystalline polymorphs thereof. In embodiments, the above described solid state forms, particularly crystalline complexes of Mirdametinib and crystalline polymorphs of Mirdametinib salts are used to prepare oral dosage forms of Mirdametinib.

The present disclosure also encompasses the use of the solid state forms, particularly crystalline complexes of Mirdametinib of the present disclosure, for the preparation of pharmaceutical compositions of crystalline complexes of Mirdametinib, particularly oral dosage forms.

The present disclosure includes processes for preparing the above mentioned pharmaceutical compositions. The processes include combining any one or a combination of the solid-state forms, particularly crystalline complexes of Mirdametinib of the present disclosure with at least one pharmaceutically acceptable excipient.

Pharmaceutical combinations or formulations of the present disclosure contain any one or a combination of the solid state forms of crystalline complexes of Mirdametinib of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Mirdametinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Mirdametinib or Mirdametinib complex, such as Mirdametinib: oxalic acid complex, can be administered. Mirdametinib may be formulated for administration to a mammal, in embodiments to a human, by injection or as ophthalmic solution for topical administration. Mirdametinib can be formulated, for example, as a viscous liquid solution or suspension, such as a clear solution, for injection or as ophthalmic solution for topical administration. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms, particularly Mirdametinib and the pharmaceutical compositions and/or formulations of Mirdametinib of the present disclosure can be used as medicaments, in embodiments for the treatment of patients with Neurofibromatosis Type 1 Associated Plexiform Neurofibromas, for the treatment of patients with low-grade gliomas, or for the treatment of patients with: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably for the treatment of patients with Neurofibromatosis Type 1 Associated Plexiform Neurofibromas or low-grade gliomas.

The present disclosure also provides methods of treating of patients with Neurofibromatosis Type 1 Associated Plexiform Neurofibromas, for the treatment of patients with low-grade gliomas, or for the treatment of patients with: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably for the treatment of patients with Neurofibromatosis Type 1 Associated Plexiform Neurofibromas or low-grade gliomas.

by administering a therapeutically effective amount of any one or a combination of the solid state forms, particularly Mirdametinib of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

Powder X-ray Diffraction (“XRPD”) method

A sample, after being powdered in a mortar and pestle, is applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 20) detector. Scanning parameters: angle range: 3-40 deg., step size 0.0167, time per step 37 s, continuous scan.

### Fourier-Transform Infrared Spectroscopy (“FTIR”)

FTIR spectra were recorded on a Nicolet 6700 interferometer between 4000 cm-1 and 650 cm-1 with resolution of 4 cm-1, in ATR technique.

### Gas Chromatography (“GC”)

GC data is obtained on Agilent 7890A or equivalent instrument with FID detector on a column DB-624, 30 m×0.53 mm, 3 μm.

### High-Performance Liquid Chromatography (“HPLC”)

HPLC data is obtained on Waters Acquity I class or equivalent instrument with PDA detector on a column YMC Triart C18 100 mm×2.0 mm, 1.9 μm.

Mobile phase A: 0.1% H3PO4

Mobile phase B: Acetonitrile

### EXAMPLES

### Preparation of Starting Materials

Mirdametinib can be prepared according to methods known from the literature, for example according to the disclosure in International Publications Nos. WO 200206213 or WO 2005040098; or U.S. Pat. No. 11,084,780.

### Example 1: Preparation of Mirdametinib Oxalic Acid Co-Crystal, Form A

Mirdametinib (0.53 grams; 1.1 mmol) was dissolved in i-propyl acetate (2.5 mL) at temperature of about 60° C. Oxalic acid (0.15 grams; 1.6 mmol) was added to the solution and the resulting mixture was stirred for 15 minutes. The heating was discontinued, and the solution was left to cool to room temperature, and was stirred for 3 days. The obtained solid was isolated by vacuum filtration over a black ribbon filter paper and a sample was analyzed by XRPD, a characteristic XRPD pattern is shown in FIG. 1. Mirdametinib Oxalic acid co-crystal form A was obtained.

### Example 2: Preparation of Mirdametinib Oxalic Acid Co-Crystal (2:1), Form A

Mirdametinib (3.0 grams; 6.2 mmol) was dissolved in ethyl acetate (6 mL) at temperature of about 50° C. Oxalic acid (0.56 grams; 6.2 mmol) was added to the solution and the resulting mixture was stirred for 30 minutes. The heating was discontinued, and the solution was left to cool to room temperature. Crystallization occurred at temperature of about 35° C. during cooling. Additional amount of ethyl acetate (14 mL) was added at room temperature and stirred for 1 hour. The suspension was further cooled at 0° C. in an ice bath and stirred for 1 hour. The obtained solid was isolated by vacuum filtration over a black ribbon filter paper and a sample was analyzed by XRPD. Mirdametinib oxalic acid co-crystal form A was obtained.

HPLC purity (area %): 99.73%

Assay (Mirdametinib): 91.0% (theor. value 91.4%)

### Example 3—Stability Studies

### Stability to Compression

A sample of Mirdametinib oxalic acid co-crystal Form A was subjected to a pressure of 2 tons for 2 minutes (Atlas® Autopress hydraulic press, set to 2 tons). XRPD analysis showed that Mirdametinib oxalic acid co-crystal Form A was resistant to amorphization when exposed to mechanical stress.

### Thermal Stability

Samples of Mirdametinib oxalic acid co-crystal Form A (100 mg) and Form IV were heated in an oven at 100° C. for 30 minutes. The resulting solids were analyzed by XRPD, and the results are shown in the table below:

| Starting material | XRPD after heating |
|---|---|
| Mirdametinib form IV | form IV + form I |
| Mirdametinib oxalic acid co-crystal form A | Form A |

The results indicate that Mirdametinib oxalic acid co-crystal Form A is surprisingly highly stable compared with Form IV, showing no conversion to other polymorph forms.

Further aspects and embodiments of the present disclosure are set out in the numbered clauses List A below:

### Clauses—List A:

1. Crystalline Mirdametinib: oxalic acid.
2. Crystalline Mirdametinib: oxalic acid which is a co-crystal.
3. A crystalline product according to Clause 1 or Clause 2, designated form A, which is characterized by data selected from one or more of the following:
   a. an X-ray powder diffraction pattern having peaks at 10.5, 13.6, 15.3, 21.2 and 22.2 degrees 2-theta±0.2 degrees 2-theta;
   b. an XRPD pattern as depicted in FIG. 1; and
   c. combinations of these data.
4. A crystalline product according to any of Clauses 1, 2 or 3, designated form A, characterized by the XRPD pattern having peaks at 10.5, 13.6, 15.3, 21.2 and 22.2 degrees 2-theta±0.2 degrees 2-theta, and also having one, two, three, four or five additional peaks selected from 5.1, 11.1, 24.3, 26.4 and 31.5 degrees two theta±0.2 degrees two theta.
5. A crystalline product according to any of Clauses 1, 2, 3, or 4, designated form A, wherein the crystalline form is an anhydrous form.
6. A crystalline product according to any of Clauses 1, 2, 3, 4 or 5, designated form A, which contains: no more than about 20%, no more than about 10%, no more than about 5%, no more than about 2%, no more than about 1% or about 0% of any other crystalline forms of Mirdametinib: oxalic acid or crystalline Mirdametinib oxalate.
7. A crystalline product according to any of Clauses 1, 2, 3, 4, 5 or 6, designated form A, which contains: no more than about 20%, no more than about 10%, no more than about 5%, no more than about 2%, no more than about 1% or about 0% of amorphous Mirdametinib: oxalic acid or crystalline Mirdametinib oxalate.
8. A pharmaceutical composition comprising a crystalline product according to any of Clauses 1-7, and at least one pharmaceutically acceptable excipient.
9. Use of a crystalline product according to any of Clauses 1-7 for the preparation of a pharmaceutical composition and/or formulation, preferably wherein the pharmaceutical formulation is an oral formulation; for example a tablet, a capsule, etc.
10. A process for preparing the pharmaceutical composition according to Clause 8, comprising combining a crystalline product according to any of Clauses 1-7 with at least one pharmaceutically acceptable excipient.
11. A crystalline product according to any of Clauses 1-7, or a pharmaceutical composition according to Clause 8, for use as a medicament.
12. A crystalline product according to any of claims 1-7, or a pharmaceutical composition according to claim 8, for use in the treatment of patients with Neurofibromatosis Type 1 Associated Plexiform Neurofibromas and for the treatment of patients with low-grade gliomas, or for the treatment of patients with: metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably for the treatment of patients with Neurofibromatosis Type 1 Associated Plexiform Neurofibromas or low-grade gliomas.
13. A method of treating Neurofibromatosis Type 1 Associated Plexiform Neurofibromas, low-grade gliomas, metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, or non-ocular melanoma; and preferably Neurofibromatosis Type 1 Associated Plexiform Neurofibromas or low-grade gliomas, comprising administering a therapeutically effective amount of a crystalline product according to any of Clauses 1-7, or a pharmaceutical composition according to Clause 8, to a subject in need of the treatment.
14. Use of a crystalline product according to any of Clauses 1-7, in the preparation of another solid-state form of Mirdametinib or another co-crystal of Mirdametinib.
15. A process for preparing a solid-state form of Mirdametinib or co-crystal of Mirdametinib comprising preparing any one or a combination of a crystalline product according to any one of Clauses 1-7, and converting it to another a solid state form thereof.

Further aspects and embodiments of the present disclosure are set out in the numbered clauses List B below:

### Clauses—List B:

1. Crystalline Mirdametinib: oxalic acid.
2. Crystalline Mirdametinib: oxalic acid which is a co-crystal.
3. A crystalline product according to Clause 1 or Clause 2, designated form A, which is characterized by data selected from one or more of the following:
   a. an X-ray powder diffraction pattern having peaks at 10.5, 13.6, 15.3, 21.2 and 22.2 degrees 2-theta±0.2 degrees 2-theta;
   b. an XRPD pattern as depicted in FIG. 1;
   c. a solid state ¹³C NMR spectrum with characteristic peaks at 159.5, 134.1, 123.7 and 77.8 ppm±0.2 ppm;
   d. a solid state ¹³C NMR spectrum as depicted in any of FIG. 3a, 3b or 3c; and
   e. combinations of these data.
4. A crystalline product according to any of Clauses 1, 2 or 3, designated form A, characterized by the XRPD pattern having peaks at 10.5, 13.6, 15.3, 21.2 and 22.2 degrees 2-theta±0.2 degrees 2-theta, and also having one, two, three, four or five additional peaks selected from 5.1, 11.1, 24.3, 26.4 and 31.5 degrees two theta±0.2 degrees two theta.
5. A crystalline product according to any of Clauses 1, 2, 3 or 4, designated form A, characterized by a solid state ¹³C NMR spectrum with characteristic peaks at 159.5, 134.1, 123.7 and 77.8 ppm±0.2 ppm and also having the following chemical shift absolute differences from reference peak at 61.8 ppm±1 ppm: 97.7, 72.3, 61.9 and 16.0 ppm±0.1
6. A crystalline product according to any of Clauses 1, 2, 3, 4 or 5, designated form A, characterized by a solid state ¹³C NMR spectrum with characteristic peaks at 159.5, 134.1, 123.7 and 77.8 ppm±0.2 ppm.
7. A crystalline product according to any of Clauses 1, 2, 3, 4, 5 or 6, designated form A, characterized by FTIR spectrum having peaks at 1710, 1498, 1190 and 1292+4 cm-1; or by a FTIR spectrum substantially as depicted in FIG. 2; and combinations of these data.
8. A crystalline product according to any of Clauses 1, 2, 3, 4, 5, 6, or 7, designated form A, wherein the crystalline form is an anhydrous form.
9. A crystalline product according to any of Clauses 1, 2, 3, 4, 5, 6, 7 or 8, designated form A, which contains: no more than about 20%, no more than about 10%, no more than about 5%, no more than about 2%, no more than about 1% or about 0% of any other crystalline forms of Mirdametinib: oxalic acid or crystalline Mirdametinib oxalate.
10. A crystalline product according to any of Clauses 1, 2, 3, 4, 5, 6, 7, 8 or 9, designated form A, which contains: no more than about 20%, no more than about 10%, no more than about 5%, no more than about 2%, no more than about 1% or about 0% of amorphous Mirdametinib: oxalic acid or crystalline Mirdametinib oxalate.
11. A pharmaceutical composition comprising a crystalline product according to any of Clauses 1-10, and at least one pharmaceutically acceptable excipient.
12. Use of a crystalline product according to any of Clauses 1-10 for the preparation of a pharmaceutical composition and/or formulation, preferably wherein the pharmaceutical formulation is an oral formulation; for example a tablet, a capsule, etc.
13. A process for preparing the pharmaceutical composition according to Clause 11, comprising combining a crystalline product according to any of Clauses 1-10 with at least one pharmaceutically acceptable excipient.
14. A crystalline product according to any of Clauses 1-10, or a pharmaceutical composition according to Clause 11, for use as a medicament.
15. A crystalline product according to any of claims 1-10, or a pharmaceutical composition according to claim 11, for use in the treatment of patients with Neurofibromatosis Type 1 Associated Plexiform Neurofibromas, low-grade gliomas, metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably to a subject suffering from Neurofibromatosis Type 1 Associated Plexiform Neurofibromas, or low-grade gliomas.
16. A method of treating: Neurofibromatosis Type 1 Associated Plexiform Neurofibromas, low-grade gliomas, metastatic breast cancer, particularly metastatic breast cancer with MAPK-mediated resistance, MAPK-mutant solid tumours, MEK1/2 mutant solid tumours, non-small cell lung cancer, colorectal cancer, Langerhans cell histiocytosis, and/or non-ocular melanoma; and preferably Neurofibromatosis Type 1 Associated Plexiform Neurofibromas, or low-grade gliomas, comprising administering a therapeutically effective amount of a crystalline product according to any of Clauses 1-10, or a pharmaceutical composition according to Clause 11, to a subject in need of the treatment.
17. Use of a crystalline product according to any of Clauses 1-10, in the preparation of another solid state form of Mirdametinib or another co-crystal of Mirdametinib.
18. A process for preparing a solid state form of Mirdametinib or co-crystal of Mirdametinib comprising preparing any one or a combination of a crystalline product according to any one of Clauses 1-10, and converting it to another a solid state form thereof.

1. A crystalline Mirdametinib: oxalic acid.

2. The crystalline Mirdametinib according to claim 1, wherein the oxalic acid is a co-crystal.

3. The crystalline Mirdametinib according to claim 1, which is characterized by data selected from one or more of the following:
a) an X-ray powder diffraction pattern (XRPD) having peaks at 10.5, 13.6, 15.3, 21.2 and 22.2 degrees 2-theta±0.2 degrees 2-theta;
b) an XRPD pattern as depicted in FIG. 1;
c) a solid state ¹³C NMR spectrum with characteristic peaks at 159.5, 134.1, 123.7 and 77.8 ppm±0.2 ppm;
d) a solid state ¹³C NMR spectrum as depicted in any of FIG. 3a, 3b or 3c; and
e) combinations of these data.

4. The crystalline Mirdametinib product according to claim 1, designated form A, characterized by an XRPD pattern having peaks at 10.5, 13.6, 15.3, 21.2 and 22.2 degrees 2-theta±0.2 degrees 2-theta, and also having one, two, three, four or five additional peaks selected from 5.1, 11.1, 24.3, 26.4 and 31.5 degrees two theta±0.2 degrees two theta.

5. The crystalline Mirdametinib according to claim 1, designated form A, characterized by a solid state ¹³C NMR spectrum with characteristic peaks at 159.5, 134.1, 123.7 and 77.8 ppm±0.2 ppm.

6. The crystalline Mirdametinib according to claim 1, designated form A, characterized by a solid state ¹³C NMR spectrum with characteristic peaks at 159.5, 134.1, 123.7 and 77.8 ppm±0.2 ppm, and also having the following chemical shift absolute differences from reference peak at 61.8 ppm±1 ppm: 97.7, 72.3, 61.9 and 16.0 ppm±1 ppm.

7. The crystalline Mirdametinib product according to claim 1, designated form A, characterized by FTIR spectrum having peaks at 1710, 1498, 1190 and 1292±4 cm-1; or by a FTIR spectrum substantially as depicted in FIG. 2.

8. The crystalline Mirdametinib according to claim 1, wherein the crystalline form is an anhydrous form.

9. The crystalline Mirdametinib according to claim 1, designated form A, which contains: no more than about 20%, no more than about 10%, no more than about 5%, no more than about 2%, no more than about 1% or about 0% of any other crystalline forms of Mirdametinib: oxalic acid or crystalline Mirdametinib oxalate.

10. The crystalline Mirdametinib according to claim 1, designated form A, which contains: no more than about 20%, no more than about 10%, no more than about 5%, no more than about 2%, no more than about 1% or about 0% of amorphous Mirdametinib: oxalic acid or amorphous Mirdametinib oxalate.

11. A pharmaceutical composition comprising the crystalline Mirdametinib according to claim 1, and at least one pharmaceutically acceptable excipient.

12. A method comprising:
preparing of a pharmaceutical composition including the Use of a crystalline Mirdametinib according to claim 1 wherein the pharmaceutical composition is at least one of an oral formulation, a tablet, or a capsule.

13. A process for preparing a pharmaceutical composition comprising the crystalline Mirdametinib according to claim 1, the process comprising combining the crystalline Mirdametinib according to claim 1 with at least one pharmaceutically acceptable excipient.

14. A method of treatment comprising:
administering to a subject in need of the treatment a therapeutically effective amount of the crystalline Mirdametinib according to claim 1 as a medicament to a subject in need of the treatment.

15. A method of treatment comprising:
administering to a subject in need of the treatment a therapeutically effective amount of the crystalline Mirdametinib according to claim 1 to treat at least one of Neurofibromatosis Type 1 Associated Plexiform Neurofibromas, low-grade gliomas, metastatic breast cancer, metastatic breast cancer with MAPK-mediated resistance, MAPK-mutant solid tumours, MEK1/2 mutant solid tumours, non-small cell lung cancer, colorectal cancer, Langerhans cell histiocytosis, non-ocular melanoma, Neurofibromatosis Type 1 Associated Plexiform Neurofibromas, or low-grade gliomas.

16. (canceled)

17. A method comprising:
preparing another solid-state form of Mirdametinib or another co-crystal of Mirdametinib from the crystalline Mirdametinib according to claim 1.

18. A process for preparing a solid-state form of Mirdametinib or co-crystal of Mirdametinib comprising preparing any one or a combination of a crystalline Mirdametinib according to claim 1, and converting the same to another a solid state form thereof.
